(19) United States
(12) Patent Application Publication
(10) Pub. No.: US 2003/0223978 A1
(43) Pub. Date: Dec. 4, 2003

(54) Conjugates of an AMF ligand and a cytotoxic molecule for use in cancer therapy

(76) Inventor: Ivan R. Nabi, Montreal (CA)

Correspondence Address: OGILVY RENAULT, 1981 MCGILL COLLEGE AVENUE, SUITE 1600, MONTREAL, QC, H3A2Y3, CA

(21) Appl. No.: 10/366,319

(22) Filed: Feb. 14, 2003

Related U.S. Application Data
(63) Continuation-in-part of application No. 09/700,844, filed on Feb. 8, 2001, now abandoned.
Application No. 09/700,844 is a 371 of international application No. PCT/CA99/00438, filed on May 13, 1999.

(30) Foreign Application Priority Data
May 22, 1998 (CA) 2,238,257

(51) Int. Cl. A61K 38/54

(52) U.S. Cl. 424/94.3

(57) ABSTRACT

The present invention relates to a therapeutical conjugate to specifically kill motile cells, which comprises a first molecule which binds to autocrine motility factor receptor (AMF-R) attached to a second toxic molecule to kill said motile cells.

### RELATED APPLICATIONS

This is a continuation-in-part of application Ser. No. 09/700,844 filed on Feb. 8, 2001. The entire content of application Ser. No. 09/700,844 is hereby incorporated by reference.

### BACKGROUND OF THE INVENTION

(a) Field of the Invention

The invention relates to the use of the endocytosis or internalization of autocrine motility factor receptor (AMF-R) as a means to target motile cells, such as metastatic tumor cells. The invention also relates to AMF-conjugates for therapeutical treatment.

(b) Description of Prior Art

Expression of autocrine motility factor receptor (AMF-R) is associated with the acquisition of motile and metastatic properties by tumor cells (Nabi, I. R. et al. (1992) Cancer Met. Rev. 11, 5-20; Silletti, S. et al. (1996) Am. J. Pathol. 148, 1649-1660). AMF-R is a cell surface receptor which mediates motility stimulation by its 55 kD polypeptide ligand, AMF, recently shown to be homologous to phosphohexose isomerase. Expression of AMF-R has been shown to be associated with tumor malignancy and inversely with patient survival associating AMF-R expression with poor prognosis. AMF-R internalizes its ligand via both clathrin-dependent and clathrin-independent pathways.

Transduction of the AMF motility signal occurs via receptor phosphorylation, a pertussis-toxin sensitive G-protein, inositol phosphate production, tyrosine kinase and protein kinase C activation and production of the lipoxygenase metabolite 12-HETE (Silletti, S. et al. (1996) Am. J.

Pathol. 148, 1649-1660). AMF-R is localized not only to the plasma membrane but also to an intracellular tubular organelle, the AMF-R tubule (Nabi, I. R. et al. (1992) Cancer Met. Rev. 11, 5-20; Benlimame, N. et al. (1995) J. Cell Biol. 129, 459-471). AMF-R tubules are distinct from endosomes and lysosomes; by post-embedding immunoelectron microscopy AMF-R is present primarily in smooth tubules which extend from ribosome-studded cisternae however AMF-R tubules do not colocalize with ERGIC-53, a marker for the ER-Golgi intermediate compartment (Benlimame, N. et al. (1995) J. Cell Biol. 129, 459-471; Wang et al., 1997). Following treatment with ilimaquinone, AMF-R tubules acquire a fenestrated morphology typical of smooth ER suggesting that the AMF-R tubule is a distinct smooth subdomain of the endoplasmic reticulum (Wang, H. -J. et al. (1997) J. Cell Sci. 110, 3043-3053). The intracellular distribution of this cell surface receptor to smooth ER implicates AMF-R recycling in its function in cell motility and tumor cell metastasis.

Anti-receptor antibodies are not necessarily endocytosed. Early studies of the transferrin receptor (TfR), that constitutively endocytoses its ligand, transferrin, showed that while monovalent anti-TfR antibodies did not affect TfR expression at the cell surface, divalent antibodies induced TfR downregulation while multivalent IgM anti-TfR induced accumulation of antibody-complexed receptor on the cell surface preventing internalization (Lesley et al., Exp Cell Res. 182:215-33, 1989). This represents only one study however it is clear that different antibodies can have different effects on cell surface receptors and that antibody binding does not necessarily result in receptor internalization. Interestingly, addition of the multivalent IgM anti-TfR antibody induced the formation of a granular distribution of cell surface antibody-receptor complexes and their consequent capping.

Moreover, endocytosis of anti-receptor antibodies does not necessarily mean that the ligand is endocytosed. Antibodies to ErbB2 are internalized, including monovalent antibodies and, to a lesser extent, the growth inhibiting herceptin anti-ErbB2 antibody (Neve et al., Biochem Biophys Res Commun. 280:274-9, 2001). Furthermore, while the ErB2 ligand EGF induces endocytosis of EGF-R homodimers it does not induce internalization of EGF-R/ERbB2 heterodimers (Wang et al., Mol Biol Cell. 10:1621-36, 1999).

AMF internalization is not necessarily required for its motility stimulating activity. The fact that anti-AMF-R mimics AMF motility stimulating ability and that anti-AMF-R competes with AMF for the same motility activating epitope (Nabi et al., Cancer Research 50:409-414,1990) does not necessarily mean that they follow the same endocytic pathway. AMF is internalized via both a clathrin-mediated pathway to multivesicular bodies and a caveolae-mediated pathway to the smooth ER (Benlimame et al., Molecular Biology of the Cell 9:1773-1786, 1998; Le et al., Journal of Cell Science 113:3227-3240, 2000; Le et al., Journal of Biological Chemistry 277:3371-3379, 2002; Le and Nabi, 2003, J. Cell Science, In Press).

Anti-AMF-R antibodies are not necessarily endocytosed and certainly do not follow the same endocytic pathway as AMF. Early studies of anti-AMF-R incubation with cells at 37° C. revealed the association of anti-AMF-R with large cell-associated clumps that were interpreted to be internalized antibody-receptor complexes (Nabi et al., Cancer Metastasis Reviews 11:5-20, 1992). However, direct evidence to indicate that these complexes were indeed intracellular and therefore endocytosed was not presented. These complexes could very well be cell surface aggregates due to complexing of cell surface AMF-R by IgM anti-AMF-R antibody as described for IgM antir TfR (Lesley et al., Exp Cell Res. 182:215-33, 1989). It was subsequently published that anti-AMF-R antibody was internalized to AMF-R positive tubules (Silletti et al., Cancer Research, 54:5752-5756, 1994). However, these data were not reproducible and as shown in FIG. 2, the addition of anti-AMF-R antibody to either NIH-3T3 fibroblasts or HeLa cells resulted in the formation of large clumps, as previously reported (Nabi et al., Cancer Metastasis Review 11:5-20, 1992), that appear in this case to be cell surface associated. This distribution contrasted with the punctuate distribution of anti-AMF-R added to cells at 4° C. that correspond to the true cell distribution of the protein (FIG. 2).

It would then be highly desirable to be provided with a means to target motile cells, such as metastatic tumor cells.

### SUMMARY OF THE INVENTION

One aim of the present invention is to provide a means to target motile cells, such as metastatic tumor cells.

Another aim of the present invention is to provide the use of the endocytosis or internalization of autocrine motility factor receptor (AMF-R) as a means to target motile cells, such as metastatic tumor cells.

In accordance with the present invention, it is demonstrated that AMF-R is concentrated at the cell surface within smooth plasmalemmal vesicles or caveolae and that AMF is internalized via a non-clathrin pathway to intracellular smooth ER tubules. The results of the present invention identify an AMR-R-mediated clathrin-independent internalization pathway to the endoplasmic reticulum which may be implicated in AMF-R function in tumor cell motility and metastasis.

In accordance with the present invention there is provided a therapeutical conjugate to specifically kill motile cells, which comprises a first molecule which binds to autocrine motility factor receptor (AMF-R) attached to a second toxic molecule to kill said motile cells, such as metastatic tumor cells.

The preferred first molecule is AMF.

The said second molecule includes, without limitation, plant toxins, bacterial toxins, fungal toxins, drugs, and enzymes for treating prodrugs.

The preferred plant toxin includes, without limitation, ricin, abrin, modeccin, viscumin, pokeweed antiviral protein (PAP), saporin, gelonin, momoridin, trichosanthin, barley toxin, and bryodin.

The preferred bacterial toxin includes, without limitation, pseudomonas exotoxin (PE), and diphtheria toxin.

The preferred fungal toxin includes, without limitation, α-sarcin, and restrictocin.

The preferred drug includes, without limitation, doxorubicin, 2-pyrrolinodoxorubicin, daunarubicin, methotrexate, neocarzinostatin, mitomycin C, calicheamicin, and vinca alkaloids.

The preferred enzyme includes, without limitation, carboxypeptidase, and alkaline phosphatase.

In accordance with the present invention there is also provided the use of internalization of autocrine motility factor receptor (AMF-R) to target motile cells, which comprises at least one molecule which binds to autocrine motility factor receptor (AMF-R) and is internalized by motile cells, such as metastatic tumor cells.

In accordance with the present invention there is also provided a method to specifically kill cancer cells in vitro and/or in vivo, which comprises administering an effective amount of the conjugate of the present invention.

The cells in vitro may be leukemias purging cells whereas the cells in vivo may be metastatic tumor cells.

All references herein are hereby incorporated by reference.

### BRIEF DESCRIPTION OF THE DRAWINGS

FIG. 1 illustrates the electron microscopic localization of AMF-R in NIH-3T3 fibroblasts and HeLa cells;

FIG. 2 illustrates the colocalization of AMF-R and caveolin by confocal microscopy;

FIG. 3 illustrates bAMF and anti-AMF-R mAb colocalize on the cell surface;

FIG. 4 illustrates the internalization of bAMF to AMF-R tubules;

FIG. 5 illustrates the localization of internalized bAMF to AMF-R tubules by confocal microscopy;

FIG. 6 illustrates the electron microscopy of the internalization pathway of bAMF;

FIG. 7 illustrates G25 Sephadex™ chromatography of AMF-doxorubicin mixture following conjugation with 0.02% glutaraldehyde;

FIG. 8 illustrates the cell toxicity of a AMF-doxorubicin conjugate in accordance with one embodiment of the present invention.

### DETAILED DESCRIPTION OF THE INVENTION

Autocrine motility factor receptor (AMF-R) is a cell surface receptor which is also localized to a smooth subdomain of the endoplasmic reticulum (ER), the AMF-R tubule. By post-embedding immunoelectron microscopy, AMF-R concentrates within smooth plasmalemmal vesicles or caveolae in both NIH-3T3 fibroblasts and HeLa cells. By confocal microscopy, cell surface AMF-R labeled by the addition of anti-AMF-R antibody to viable cells at 4° C. exhibits partial colocalization with caveolin confirming the localization of cell surface AMF-R to caveolae. Labeling of cell surface AMF-R by either anti-AMF-R antibody or biotinylated AMF (bAMF) exhibits extensive colocalization and after a pulse of 1-2 hours at 37° C., bAMF accumulates in densely labeled perinuclear structures as well as fainter tubular structures which colocalize with AMF-R tubules. After a subsequent 2-4 hour chase bAMF is localized predominantly to AMF-R tubules. Cytoplasmic acidification, blocking clathrin-mediated endocytosis, results in the essentially exclusive distribution of internalized bAMF to AMF-R tubules. By confocal microscopy, the tubular structures labeled by internalized bAMF show complete colocalization with AMF-R tubules. bAMF internalized in the presence of a 10-fold excess of unlabeled AMF labels perinuclear punctate structures, which are therefore the product of fluid phase endocytosis, but not AMF-R tubules demonstrating that bAMF targeting to AMF-R tubules occurs via a receptor-mediated pathway. By electron microscopy, bAMF internalized for 10 minutes is located to cell surface caveolae and after 30 minutes is present within smooth and rough ER tubules. AMF is therefore internalized via a receptor-mediated clathrin-independent pathway to smooth endoplasmic reticulum. The steady state localization of AMF-R to caveolae implicates these cell surface invaginations in AMF-R endocytosis.

Materials and Methods

Cells and Cell Culture

NIH-3T3 fibroblasts obtained from the ATCC were cloned and a highly spread clone was used for these studies. HeLa and NIH-3T3 cells were grown in an air-5% CO2 incubator at constant humidity in Dulbecco's minimum essential medium (DMEM) containing non-essential amino acids, vitamins, glutamine and a penicillin-streptomycin antibiotic mixture (Gibco, Burlington, Ontario) supplemented with 5% fetal calf serum (Immunocorp, Montreal, Quebec) for HeLa or 10% calf serum (Gibco, Burlington, Ontario) for NIH-3T3 cells.

Antibodies and Chemicals

Monoclonal antibody against AMF-R was used in the form of concentrated hybridoma supernatant (Nabi, I. R. et al. (1992) Cancer Met. Rev. 11, 5-20). Rabbit anti-caveolin polyclonal antibody was purchased from Transduction Laboratories (Lexington, Ky.), rabbit anti-biotin antibody from Sigma (St. Louis, Mo.), and rat anti-LAMP-1 from the Developmental Studies Hybridoma Bank (University of Iowa, Iowa City). Secondary antibodies conjugated to either fluorescein, Texas Red or 12 nm gold particles and streptavidin conjugated to fluorescein or Texas Red were purchased from Jackson Immunoresearch Laboratories (West Grove, Pa.). Texas Red conjugated human diferric transferrin was kindly provided by Dr. Tim McGraw (Columbia University, New York, N.Y.). Streptavidin conjugated to 10 nm gold particles was purchased from Sigma. The secondary antibodies were designed for use in multiple labeling studies and no interspecies cross-reactivity was detected. To detect antibodies to AMF-R, secondary antibodies specific for the p chain of rat IgM were used.

Rabbit phosphohexose isomerase was purchased from Sigma and biotinylated with NHS-LC-biotin (Pierce, Rockford, Ill.) according to the manufacturer's instructions. To assess its purity, biotinylated phosphohexose isomerase was separated by SDS-PAGE, transferred to nitrocellulose, probed with horseradish peroxidase conjugated streptavidin (Jackson Immunoresearch Laboratories) and revealed by chemiluminescence.

Immunofluorescence

Cells were plated on glass cover slips 2 days prior to each experiment at a concentration of 30,000 cells/35 mm dish. For AMF-R surface labeling, the cells were incubated in DMEM minus bicarbonate supplemented with 25 mM Hepes pH 7.2 and 2.5% serum for 15 min at 4° C. prior to labeling with anti-AMF-R primary antibody or biotinylated AMF at 4° C. for 30 min. The cells were washed at 4° C. and then fixed with 3% paraformaldehyde in phosphate buffered saline (pH 7.4) supplemented with 0.1 mM Ca++ and 1 mM Mg++ (PBS/CM) for 15 min at room temperature. For caveolin labeling, after AMF-R surface labeling at 4° C. and fixation as above, the cells were permeabilized with 0.2% Triton X-100 for 10 min, then extensively washed with PBS/CM containing 1% BSA. The cells were incubated with rabbit anti-caveolin polyclonal antibodies, washed, and then incubated with FITC goat anti-rat IgM to reveal anti-AMF-R and Texas Red donkey anti-rabbit IgG to reveal anti-caveolin. Cell surface labeling with biotinylated AMF was revealed with rabbit anti-biotin antibody and fluorescent anti-rabbit secondary antibody.

For the AMF internalization studies, NIH-3T3 cells were pulsed with biotinylated AMF (˜250-500 μg/ml) and chased at 37° C. for the indicated periods of time prior to fixation by the addition of precooled (−80° C.) methanol/acetone directly to the cells. After fixation, internalized bAMF was revealed with Texas Red streptavidin and lysosomes and AMF-R tubules by anti-LAMP-1 and anti-AMF-R antibodies, respectively, followed by the corresponding FITC-conjugated secondary antibodies. Disruption of clathrin coated pits and vesicles by cytoplasmic acidification was performed essentially as previously described (Heuser, J. (1989) J. Cell Biol. 108, 401-411). NIH-3T3 cells were pretreated with acidification medium (DMEM containing 5% calf serum and 50 mM MES pH 5.5) for 15 minutes at 37° C. prior to addition of bAMF in acidification medium for one hour at 37° C. To ensure that cellular acidification blocked clathrin-mediated endocytosis, Texas Red transferrin (50 μg/ml) was added to cells in regular or acidification medium for 30 minutes at 37° C. after which the cells were fixed with 3% paraformaldehyde.

After labeling the coverslips were mounted in Airvol (Air Products and Chemicals Inc., Allentown, Pa.) and viewed in a Zeiss Axioskop fluorescent microscope equipped with a 63× Plan Apochromat objective and selective filters. Confocal microscopy was performed with the 60× Nikon Plan Apochromat objective of a dual channel BioRad 600 laser scanning confocal microscope equipped with a krypton/argon laser and the corresponding dichroic reflectors to distinguish fluorescein and Texas Red labeling. Confocal images were printed using a Polaroid TX 1500 video printer.

Electron Microscopy

Post-embedding immunolabeling for AMF-R was performed as previously described (Benlimame, N. et al. (1995) J. Cell Biol. 129, 459-471). Cells grown on petri dishes were rinsed and incubated at 37° C. in Ringer's solution for 15 minutes before fixing in Ringer's solution containing 2% paraformaldehyde and 0.2% glutaraldehyde for 30 minutes at 37° C. The fixed cells were rinsed in PBS/CM, scraped from the petri dish and collected by centrifugation. The cell pellet was post-fixed for 30 minutes with 1% osmium tetroxide in PBS/CM containing 1.5% potassium ferrocyanide (reduced osmium), dehydrated and embedded in LR-White resin. Ultra-thin sections (80 nm) were blocked with 2% BSA, 0.2% gelatin in PBS/CM for 1 hour, and then incubated at room temperature with anti-AMF-R antibody for 1 hour followed by 12 nm gold conjugated goat anti-rat antibodies for 1 hour. The sections were then stained with 5% uranyl acetate and examined in a Philips 300 electron microscope. The numerical density of gold particles associated with plasma membrane, caveolae, clathrin coated pits and vesicles, smooth tubules and vesicles, and rough ER was determined. The length of the limiting membrane of the indicated organelles was measured using a Sigma-Scan measurement system and the gold particles associated with these organelles counted. Rough ER was defined by the presence of a linear array of membrane-associated ribosomes. Smooth vesicles attached to the plasma membrane or within 100 nm of the plasma membrane were considered to be caveolae. Control labeling with non-immune rat IgM antibodies was analyzed similarly.

To follow the endocytic pathway of AMF by electron microscopy, biotinylated AMF was internalized as described for the fluorescence studies and detected by postembedding labeling with streptavidin conjugated to 10 nm gold as described above. No labeling was observed in the absence of biotinylated AMF.

Expression of AMF and AMF-R in Normal Tissue

The AMF/PHI polypeptide has been shown to be expressed in various tissues in the mouse including muscle, salivary gland, brain, liver and kidney. PHI mRNA exhibits particularly high expression in the muscle, brain and kidney of adult mouse, rat and chicken. PHI is identical to neuroleukin, which increases the survival of cultured sensory neurons and which is secreted by lectin-stimulated T cells and induces maturation of B-cells into antibody secreting cells, and to maturation factor, which induces the differentiation of human myeloid cells into monocytes. Consistent with its identity with neuroleukin and maturation factor, AMF activity has been detected in rheumatoid synovial fluid. Therefore, in addition to its enzymatic activity, the secreted PHI polypeptide functions as a neurotrophic factor, a lymphokine and a motility factor.

In the studies of AMF-R expression in bladder, colorectal, gastric, skin and esophageal cancers as well as in breast carcinoma, AMF-R was expressed not at all or at significantly reduced levels in adjacent normal tissue. Studies performed in the Nabi laboratory have shown that while AMF-R is expressed in brain, liver and lung but not kidney and muscle of young (postnatal day 5 and 12) rats, AMF-R expression is significantly reduced or absent in these tissues in the adult. In the cerebellum, AMF-R was localized to cerebellar neurons, including Purkinje cells, and may play a role in neuronal plasticity and establishment of neuronal contacts during development. Cell and tissue specific expression of AMF and AMF-R may regulate their motile activity during development; deregulation of the expression of AMF and its receptor during tumorigenesis may contribute to the acquisition of motile and invasive properties by tumor cells. The dramatically decreased expression of AMF-R in normal cells in the adult certainly supports its utility as a target for tumor therapy.

Localization of AMF-R to Cell Surface Caveolae

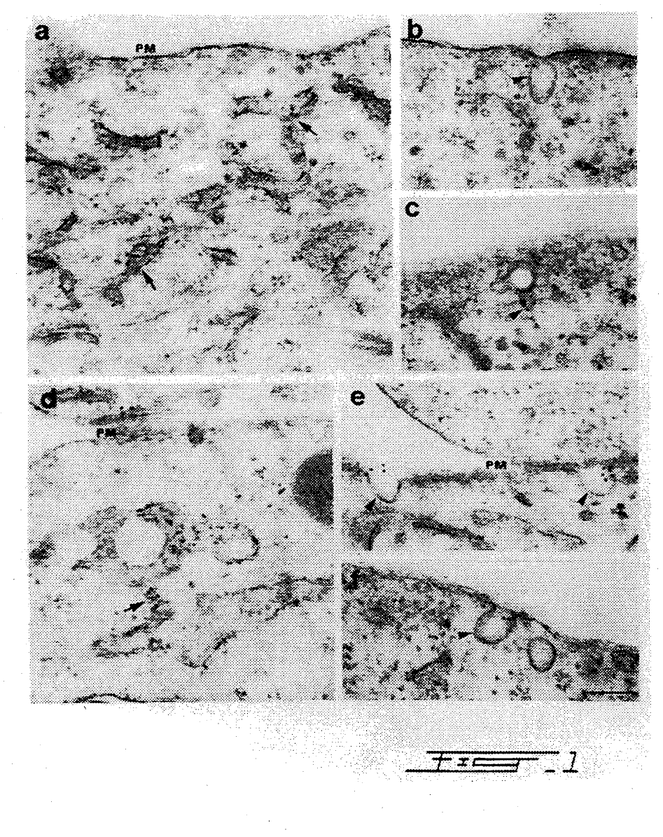

By post-embedding immunoelectron microscopy in NIH-3T3 and HeLa cells, AMF-R is primarily localized to smooth intracellular membranous tubules (FIGS. 1A, D), similar in morphology to those previously described in MDCK cells (Benlimame, N. et al. (1995) J. Cell Biol. 129, 459-471). HeLa (FIGS. 1A, B, C) and NIH-3T3 (FIGS. 1D, E, F) cells were post-embedding immunolabeled with anti-AMF-R and 12-nm gold-conjugated anti-rat IgM secondary antibodies. Typical AMF-R labeling of smooth tubules (FIGS. 1A, D, arrows) and cell surface caveolae (FIGS. 1B, C, E, F, arrowheads) is shown. PM: plasma membrane. Bar=0.2 μm.

At the cell surface, AMF-R label localizes to smooth invaginations of the plasma membrane morphologically equivalent to caveolae (FIGS. 1B, C, E, F). Quantification of the labeling revealed that the predominant AMF-R label is localized to smooth tubules and vesicles, flat regions of the plasma membrane and caveolae (Table 1).

TABLE 1
Localization of AMF-R in HeLa and NIH-3T3 cells by immunoelectron
microscopy

| | | | | | Clathrin- |
|---|---|---|---|---|---|
| | Smooth | Rough | | | coated pits |
| | tubules and | endoplasmic | Flat plasma | | and |
| | vesicles | reticulum | membrane | Caveolae | vesicles |
| HeLa | | | | | |
| AMF-R | | | | | |
| # gold particles | 660 | 34 | 147 | 52 | 3 |
| μm membrane | 328.5 | 187.5 | 245.6 | 14.6 | 19.3 |
| gold particles/μm | 2.01 ± 0.15 | 0.18 ± 0.04 | 0.60 ± 0.08 | 3.56 ± 0.53 | 0.16 ± 0.13 |
| Control | | | | | |
| # gold particles | 83 | 6 | 25 | 3 | 1 |
| μm membrane | 307.5 | 95.2 | 211.0 | 16.1 | 5.0 |
| gold particles/μm | 0.27 ± 0.08 | 0.06 ± 0.03 | 0.12 ± 0.05 | 0.19 ± 0.10 | 0.20 ± 0.21 |
| NIH-3T3 | | | | | |
| AMF-R | | | | | |
| # gold particles | 640 | 74 | 296 | 44 | 2 |
| μm membrane | 432.6 | 308.9 | 308.8 | 28.8 | 34.7 |
| gold particles/μm | 1.48 ± 0.10 | 0.24 ± 0.06 | 0.96 ± 0.10 | 1.53 ± 0.30 | 0.06 ± 0.06 |
| Control | | | | | |
| # gold particles | 33 | 7 | 10 | 4 | 2 |
| μm membrane | 303.1 | 109.8 | 138.1 | 31.7 | 6.2 |
| gold particles/μm | 0.11 ± 0.02 | 0.06 ± 0.03 | 0.07 ± 0.03 | 0.13 ± 0.06 | 0.32 ± 0.18 |

Gold particles associated with the indicated membrane organelles were counted and the density per μm membrane length determined. Control labeling was determined using a nonimmune rat IgM antibody (Benlimame, N. et al. (1995) J. Cell Biol. 129, 459-471).

While specific label was previously detected in the rough ER of MDCK cells (Benlimame, N. et al. (1995) J. Cell Biol. 129, 459-471), the density of labeling of rough ER tubules in NIH-3T3 and HeLa cells is reduced and at control levels. The density of AMF-R labeling of caveolae is equal to that of intracellular smooth tubules and vesicles in NIH-3T3 cells and greater than that of intracellular smooth tubules and vesicles in HeLa cells and essentially no AMF-R label is found within clathrin coated pits and vesicles. The density of AMF-R labeling in caveolae is increased relative to flat regions of the plasma membrane. However, based on the total number of gold particles at the plasma membrane, only 13% of cell surface AMF-R in NIH-3T3 and 26% in HeLa cells is found within caveolae.

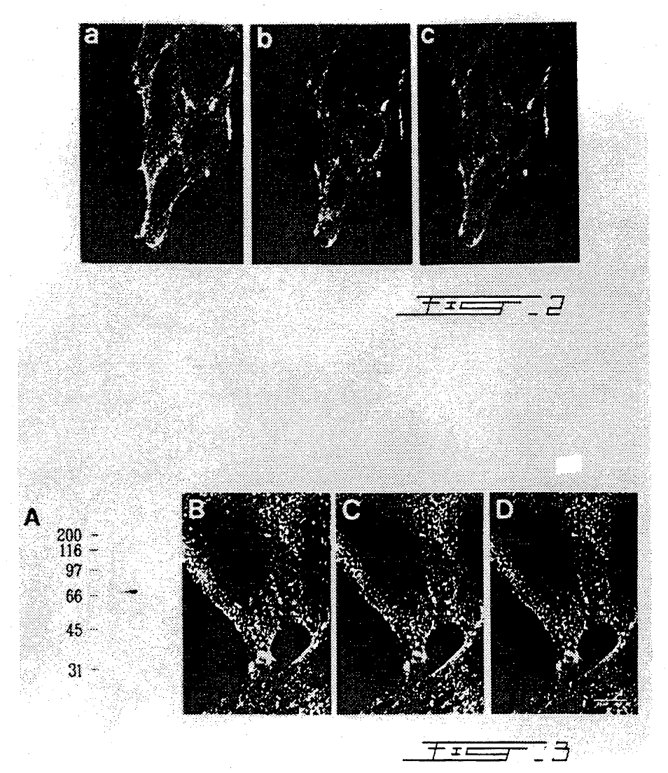

To assess whether cell surface AMF-R colocalizes with caveolin, viable NIH-3T3 cells were surface labeled for AMF-R by the addition of anti-AMF-R antibodies to viable cells at 4° C. (Nabi, I. R. et al. (1992) Cancer Met. Rev. 11, 5-20) and then double immunofluorescently labeled after fixation and permeabilization with antibodies to caveolin (FIG. 2). Viable NIH-3T3 cells were labeled for cell surface AMF-R at 4° C. (FIG. 2A) and for caveolin after fixation and permeabilization (FIG. 2B). To demonstrate the colocalization of AMF-R and caveolin, confocal images from both fluorescent channels were superimposed (FIG. 2C, AMF-R in green and caveolin in red) and colocalization appears in yellow. Bar=20 μm.

While the punctate AMF-R surface label (FIG. 2A) did not completely colocalize with the finer caveolin labeling (FIG. 2B), confocal microscopy clearly revealed distinct points and patterns labeled for both cell surface AMF-R and caveolin (FIG. 2C, yellow). Peripheral regions densely labeled for both AMF-R and caveolin were frequently observed. The partial colocalization of cell surface AMF-R with caveolin is consistent with the fact that, based on the EM data, only 13% of cell surface AMF-R was localized within the caveolae of NIH-3T3 cells.

Internalization of AMF

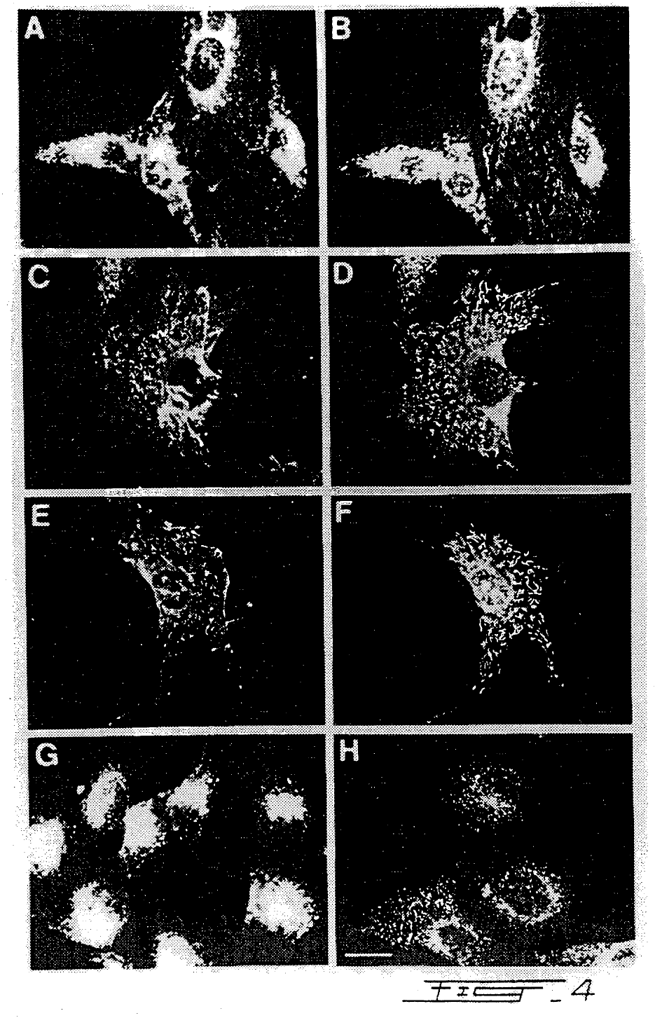

The ligand for AMF-R, AMF, is homologous to phosphohexose isomerase (Watanabe, H. et al. (1996) Cancer Res. 56, 2960-2963). Phosphohexose isomerase (referred to here as AMF) was biotinylated and after separation by SDS-PAGE revealed a single major band after revelation of the blots with streptavidin-HRP (FIG. 3A). bAMF (phosphohexose isomerase) migrated as a single band in protein blots revealed with HRP-streptavidin (FIG. 3A). Confocal imaging of cell surface labeling of viable NIH-3T3 cells at 4° C. with bAMF (FIG. 3B) or anti-AMF-R antibody (FIG. 3C). Confocal images from both fluorescent channels were superimposed (FIG. 3D; bAMF in green and AMF-R in red) and revealed a significant degree of colocalization in yellow. Bar=20 μm.

Cell surface labeling of NIH-3T3 cells by the addition of both biotinylated AMF (bAMF) (FIG. 3B) and anti-AMF-R at 4° C. (FIG. 3C) revealed a high degree of colocalization (FIG. 3D, yellow) demonstrating that AMF and antibodies to AMF-R recognize the same receptor. The presence of spots labeled exclusively with either bAMF or anti-AMF-R may be due to the fact that the two were added together and may compete for the same site.

Pulse labeling of NIH-3T3 cells with bAMF for one or two hours resulted in the ability to detect both punctate structures as well as fainter tubular structures which colocalized with AMF-R tubules (FIGS. 4A, B). NIH-3T3 cells were pulse labeled with bAMF at 37° C. for one hour (FIGS. 4A, B), for two hours and chased for 4 hours (FIGS. 4C, D) or for one hour in medium acidified to pH 5.5 to disrupt clathrin-mediated endocytosis (FIGS. 4E, F). After fixation with methanol/acetone, cells were double labeled with Texas Red-streptavidin to reveal bAMF (FIGS. 4A, C, E) and anti-AMF-R mAb and FITC-conjugated anti-rat secondary antibody to reveal AMF-R labeling (FIGS. 4B, D, F). To ensure that cellular acidification disrupted clathrin-mediated endocytosis of transferrin receptor, NIH-3T3 cells were incubated at 37° C. with Texas Red transferrin for 30 minutes in regular medium (FIG. 4G) or in medium acidified to pH 5.5 (FIG. 4H). Bar=20 μm.

Under these conditions, the extent of punctate and tubular labeling varied between cells. Fibrillar labeling of bAMF was also observed and has been determined to be localized to the cell surface. An extended chase of 2 or 4 hours after a two hour pulse resulted in decreased punctate labeling and the accumulation of bAMF labeling in tubular structures which colocalized with AMF-R tubules (FIGS. 4C, D). The vast majority of the cells exhibited predominantly intracellular tubular labeling as well as cell surface fibrillar labeling. Following treatment of cells with acidified medium (pH 5.5) and disruption of clathrin coated pits and vesicles (Heuser, J. (1989) J. Cell Biol. 108, 401-411), bAMF internalized for one hour is localized to intracellular AMF-R tubules (FIGS. 4E,F). In the acidified medium, internalized transferrin did not cluster in the perinuclear recycling compartment demonstrating that the acidification procedure did indeed disrupt clathrin-mediated endocytosis (FIGS. 4G,H). bAMF is therefore internalized via a clathrin-independent endocytic pathway to the smooth ER.

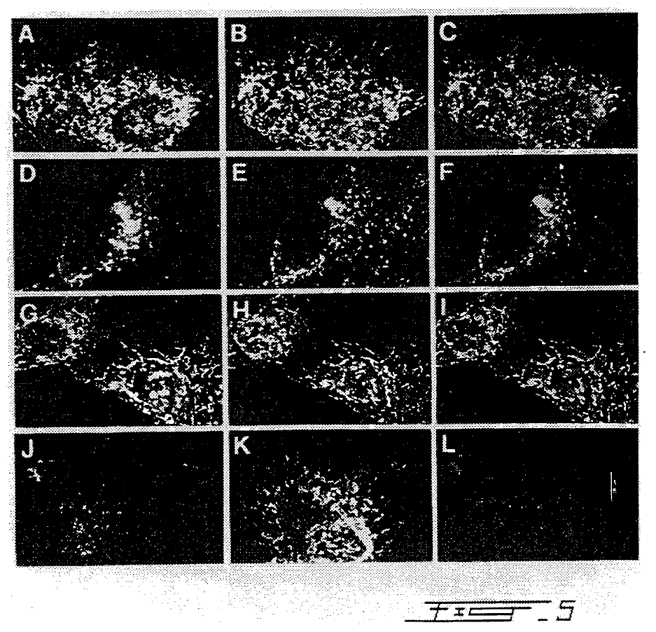

The colocalization of bAMF labeled tubules with AMF-R tubules was confirmed by confocal microscopy (FIG. 5). NIH-3T3 cells were pulse labeled with bAMF at 37° C. for one hour in regular medium (FIGS. 5A-F), for one hour in medium acidified to pH 5.5 to disrupt clathrin-mediated endocytosis (FIGS. 5G-I), or in regular medium in the presence of 10-fold excess unlabeled AMF (FIGS. 5J-L) prior to fixation with methanol/acetone. bAMF was revealed with Texas Red-streptavidin (FIGS. 5A, D, G, J), and AMF-R (FIGS. 5B, H, K) or LAMP-1 (FIG. 5E) labeled with the appropriate primary antibodies and FITC-conjugated secondary antibodies. Confocal images from both fluorescent channels were superimposed (FIGS. 5C, I, L: bAMF in red and AMF-R in green; F: bAMF in red and LAMP-1 in green) and colocalization appears in yellow. Bar=10 μm.

Following a 1 hour bAMF internalization, internalized bAMF is localized to tubular structures which colocalize with AMF-R tubules (FIGS. 5A-C) as well as to punctate structures which exhibit partial colocalization with LAMP-1 positive lysosomes (FIGS. 5D-F). As seen here, the intense punctate labeling can hide the fainter tubular labeling of bAMF in some cells (FIGS. 4A, 5D). In acidification medium, the vast majority of bAMF labeling, aside from cell surface fibrils, is localized to tubules which colocalize with AMF-R tubules (FIGS. 5G-I). bAMF internalized for 1 hour in the presence of 10-fold excess unlabeled AMF is localized only to punctate structures and no labeling of AMF-R tubules can be detected (FIGS. 5J-L). While the extent of tubular labeling of bAMF varies between cells under control conditions (FIGS. 5A,D), in the presence of excess unlabeled AMF the localization of bAMF to AMF-R tubules is never observed (FIGS. 5J-L). bAMF internalization to intracellular AMF-R tubules therefore occurs via a receptor-mediated process. The inability of excess AMF to block bAMF internalization to punctate perinuclear structures, which exhibit partial colocalization with LAMP-1 labeled lysosomes, demonstrates that this labeling is not saturable and corresponds to non-specific fluid phase uptake. The disappearance of lysosomal labeling following extended chase times (FIG. 4C, D) is therefore most likely due to lysosomal degradation of fluid phase internalized bAMF.

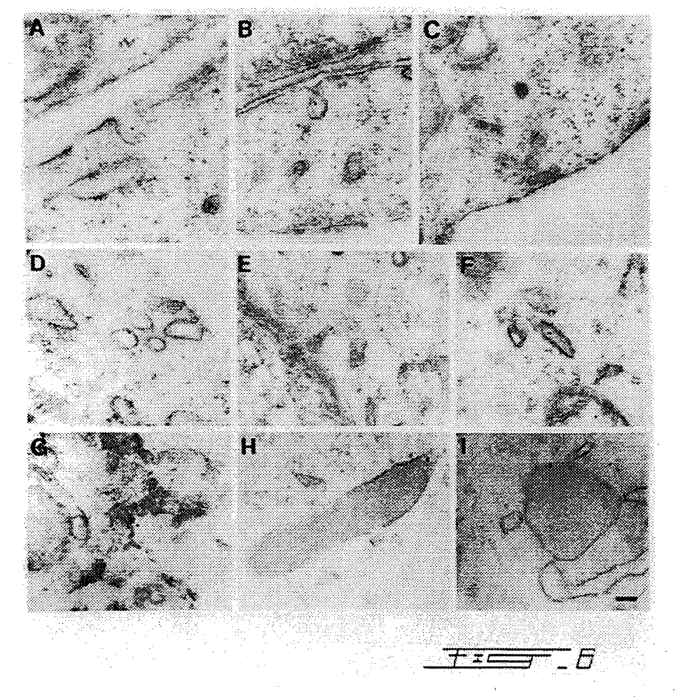

The location of biotinylated AMF internalized at 37° C. was determined by post-embedding electron microscopy with streptavidin-10 nm gold. Following a 10 minute pulse biotinylated AMF could be detected in caveolae (FIGS. 6A, B). NIH-3T3 cells were pulsed with bAMF at 37° C. for 10 (FIGS. 6A, B, H) or 30 minutes (FIGS. 6C, D, E, F, G, I). The localization of bAMF was revealed by postembedding labeling with 10 nm gold-conjugated streptavidin. After 10 minutes, bAMF is localized to cell surface caveolae (FIGS. 6A, B). After a 30 minute pulse, bAMF is localized to caveolae and smooth vesicles (FIGS. 6C, D) and also appears in intracellular membranous tubules (FIGS. 6E, F, G) including distinctive smooth (FIG. 6E) and rough (FIG. 6F) ER elements. bAMF labeling of dense lysosomal structures is also detected (FIG. 6H, I). Bar=0.1 μm.

Following a 30 minute pulse, both caveolae and intracellular smooth and rough ER elements were labeled (FIGS. 6C-G). Dense structures morphologically equivalent to lysosomes are also labeled and presumably correspond to the perinuclear structures densely labeled for internalized bAMF by immunofluorescence (FIGS. 6H, I).

AMF-R Localization to Caveolae

By post-embedding immunoelectron microscopy AMF-R is localized to morphologically identifiable caveolae as well as to smooth ER tubules (FIG. 1; Table 1). In contrast to polarized epithelial MDCK cells (Benlimame, N. et al. (1995) J. Cell Biol. 129, 459-471), labeling of rough ER tubules was not above background in either NIH-3T3 or HeLa cells indicating that AMF-R is a specific marker for smooth ER in these two cell types. The localization of AMF-R to caveolae was confirmed by the colocalization of cell surface AMF-R, labeled by the addition of anti-AMF-R to viable cells at 4° C., with caveolin by confocal fluorescence microscopy (FIG. 2). By both postembedding immunoelectron microscopy and confocal double labeling with caveolin, only a minor portion of cell surface AMF-R actually distributes to caveolae identified either morphologically or by the presence of caveolin. Based on the labeling of AMF-R by immunoelectron microscopy, only about 5% of total cellular AMF-R is actually localized to caveolae (Table 1).

Transduction of the AMF motility signal is mediated by a pertussis-toxin sensitive G protein, phosphorylation of AMF-R and both protein kinase C and tyrosine kinase activities (Silletti, S. et al. (1996) Am. J. Pathol. 148: 1649-1660). Caveolar cell surface domains have been proposed to be plasma membrane regions which serve to assemble molecules involved in receptor-mediated signal transduction, including heterodimeric G-proteins, protein kinase C and tyrosine kinases (Lisanti, M. P. et al. (1994) Trends Cell Biol. 4:231-235). The involvement of heterotrimeric G proteins as well as tyrosine kinase and protein kinase C activities in transduction of the AMF motility signal is consistent with the localization of AMF-R to cell surface caveolae.

Clathrin-Independent Internalization of AMF-R to Smooth ER Tubules

The presence of AMF-R both at the cell surface and within an intracellular ER-associated organelle suggested that the receptor recycles between these two cellular sites (Nabi, I. R. et al. (1992) Cancer Met. Rev. 11: 5-20; Benlimame, N. et al. (1995) J. Cell Biol. 129: 459-471). The fact that the density of AMF-R labeling within caveolae is equivalent to (NIH-3T3) or greater than (HeLa) that of smooth vesicles and tubules is consistent with the concentration of AMF-R within caveolae prior to vesicle budding and fusion with AMF-R tubules (Table 1).

AMF exhibits sequence identity to phosphohexose isomerase (Watanabe, H. et al. (1996) Cancer Res. 56: 2960-2963). Biotinylated phosphohexose isomerase or bAMF colocalizes with cell surface AMF-R labeled with antibodies to AMF-R at 4° C. and is endocytosed by cells at 37° C. to tubules which colocalize with smooth ER AMF-R tubules by confocal microscopy and to morphologically identifiable ER tubules by electron microscopy. bAMF internalization to smooth ER tubules is a receptor-mediated process as it can be blocked by the presence of excess unlabeled AMF. Cellular acidification, which specifically blocks clathrin mediated endocytosis, disrupts the internalization of transferrin, but not that of bAMF to AMF-R tubules, demonstrating that AMF-R is internalized to AMF-R tubules via a non-clathrin endocytic pathway. Under the conditions used in these experiments, internalization of bAMF to lysosomal structures is also observed. This lysosomal labeling is observed even in the presence of excess unlabeled AMF indicating that it is not receptor-mediated and due to fluid phase uptake. We have therefore identified a clathrin-independent AMF-R-mediated endocytic pathway which targets bAMF to the endoplasmic reticulum. The localization of AMF-R and internalized bAMF to cell surface caveolae by electron microscopy implicates these smooth invaginations of the plasma membrane in the endocytosis of AMF-R to the smooth ER subdomain for which it is a marker (Benlimame, N. et al. (1995) J. Cell Biol. 129: 459-471; Wang, H. -J. et al. (1997) J. Cell Sci. 110: 3043-3053).

Role of Caveolae in AMF-R Internalization

Whether caveolae are involved in endocytic processes in non-endothelial cells remains a controversial subject and whether clathrin-independent internalization routes involve caveolae or clathrin-coated pits without the clathrin is not clear (van Deurs, B. et al. (1993) Trends Cell Biol. 3: 249-251).

The receptor-mediated endocytosis of bAMF not to endosomes and lysosomes but to the ER certainly suggests that bAMF endocytosis is not mediated by uncoated clathrin vesicles. SV40 virus associates with caveolae and is internalized via smooth plasmalemmal vesicles to smooth tubules which are extensions of the ER (Kartenbeck, J. et al. (1989) J. Cell Biol. 109: 2721-2729). The internalization pathway of SV40 to the ER (Kartenbeck, J. et al. (1989) J. Cell Biol. 109: 2721-2729) is therefore remarkably similar to that of AMF-R described here and the localization of both AMF-R and SV40 to cell surface caveolae certainly implicates caveolae in this ER directed endocytic pathway. AMF activation of AMF-R may stimulate both transduction of the AMF motility stimulating signal and internalization of AMF-R, perhaps within the same cell surface caveolar domain.

AMF Recycling and Cell Motility

The established role of AMF-R in cell motility and metastasis implicates AMF-R internalization, and subsequent recycling to the cell surface, in the motile process (Nabi, I. R. et al. (1992) Cancer Met. Rev. 11: 5-20; Silletti, S. et al. (1996) Am. J. Pathol. 148: 1649-1660). This recycling pathway stimulated by the cytokine, AMF, may represent a motility specific membrane targeting pathway.

The present invention will be more readily understood by referring to the following examples which are given to illustrate the invention rather than to limit its scope.

### EXAMPLE I

AMF-R as a Target for Tumor Therapy

The internalization of cancer specific ligands as a means to target metastatic tumor cells has previously been demonstrated for the BR96 antigen. The monoclonal antibody BR96 is specific for the LeY polylactosamine carbohydrate antigen expressed abundantly on numerous carcinomas (Hellström, I. et al. (1990) Cancer Res. 50:2183-2190). The BR96 antibody has been shown to be internalized via coated pits to multivesicular bodies, endosomes and finally to lysosomes where it is degraded (Garrigues, J. et al. (1993) Am. J. Pathol. 142: 607-622) Toxin conjugates of this internalizing monoclonal antibody, such as BR96 doxorubicin immunoconjugates or Pseudomonas PE40 exotoxin fusion proteins, effected complete regression of xenografted human carcinomas in athymic mice (Friedman, P. N. et al. (1993) J. Immunol. 150: 3054-3061; Trail, P. A. et al. (1993) Science 261:212-215).

Based on our knowledge to date, AMF-R is an attractive target for tumor therapy because (1) its expression is specifically associated with an essential element of tumor cell metastasis, cell motility, and it exhibits minimal expression in normal cells, (2) its ligand is a common cellular protein, phosphohexose isomerase, which should not generate an immune response and which should exhibit a relatively long biological half-life, and (3) AMF is internalized by its receptor. This last point is critical: it distinguishes the use of AMF-R as a target from many cell surface tumor markers and permits the internalization of a toxic compound. Normal and tumor cells will therefore be discriminated at two levels: receptor expression and receptor internalization. It is proposed herein to prepare AMF-toxin conjugates and test their ability to kill breast cancer cells in vitro and test their toxicity in mice with the goal to establish the viability of this approach to eliminate breast tumor cells in vivo.

### EXAMPLE II

Preparation of AMF-Toxin Conjugates

PHI from mouse and human exhibit 87% homology indicating a high degree of sequence homology between species. For our studies of AMF internalization, we are currently using commercially available rabbit muscle PHI indicating that rabbit PHI is recognized by mouse AMF-R (Benlimame et al., 1998, Molec. Biol. Cell 9: 1773-1786). We will initially prepare AMF-toxin conjugates with rabbit PHI since we have demonstrated its internalization in mouse NIH-3T3 fibroblasts for convenience (Benlimame et al., 1998, Molec. Biol. Cell 9: 1773-1786), however we have available the human PHI cDNA and are in the process of generating tagged recombinant human AMF. The purpose of these studies is to assess whether AMF-toxin conjugates can be used to target tumor cells and AMF will be therefore conjugated to both the ricin α-chain and doxorubicin (DOX).

Glutaraldehyde is a non-specific crosslinker which links amine groups and has previously been used to conjugate various proteins to DOX, including transferrin which is internalized by its receptor to early endocytic compartments. This relatively simple crosslinking approach was used to generate AMF conjugated to doxorubixin. AMF-DOX is prepared by glutaraldehyde crosslinking as previously described for transferrin. The maximal glutaraldehyde concentration which does not generate high molecular weight oligomers of AMF is determined by SDS-PAGE. Using this glutaraldehyde concentration as well as moderately higher and lower concentrations, AMF is reacted with an excess molar ratio (20:1) of DOX in order to favor DOX conjugation to AMF. Reactive glutaraldehyde is neutralized with lysine and the DOX-AMF conjugate separated from free DOX by either dialysis or Sephadex G25 chromatography. The extent of DOX incorporation into the conjugate is determined by spectrophotometry at 495 nm (absorbance of DOX) after establishing concentration curves with known concentrations of free DOX. Protein concentration of the conjugate is determined by spectrophotometry at 280 nm (for AMF protein) in comparison with known concentrations of AMF and taking into consideration potential interference of DOX. Alternatively, protein concentration can be determined by densitometry of coomassie stained SDS-PAGE gels in comparison with known amounts of unconjugated AMF.

While the use of glutaraldehyde should guarantee successful crosslinking of AMF to toxin, its homofunctional nature may result in the formation of high molecular weight oligomers which may limit activity of the AMF-DOX conjugate. If necessary, we will also conjugate AMF to the ricin α-chain using the heterobifunctional crosslinker N-succinimidyl 3-(2-pyridyldithio)propionate (SPDP) which contains NH2- and SH-reactive domains and which has previously been used to form ricin-antibody conjugate immunotoxins (Hellström, I. et al. (1990) Cancer Res. 50:2183-2190). SPDP-conjugated ricin α-chain will be reduced with DTT to generate free SH sites which can attack the intact SH-reactive sites of SPDP-PHI. Crosslinking will be assessed by SDS-PAGE under reducing and non-reducing conditions; successful crosslinking of the two proteins should result in the presence of a protein band of ˜100-110 kD only under non-reducing conditions. The crosslinking will also be performed using long chain LC-SPDP which contains an internal spacer between the reactive groups thereby reducing inefficient cross-linking due to steric hindrance. Ricin conjugated AMF can be purified from free AMF by affinity chromatography on Blue sepharose CL-6B.

List of Potential Toxins/Enzymes for use in the Conjugate of the Present Invention:

Plant Toxins

ricin

abrin

modeccin

viscumin

pokeweed antiviral protein (PAP)

saporin

gelonin

momoridin

trichosanthin

barley toxin

bryodin

Bacterial Toxins

pseudomonas exotoxin (PE)

diphtheria toxin

Fungal Toxins

α-sarcin

restrictocin

Drugs

doxorubicin (also called adriamycin)

2-pyrrolinodoxorubicin (a more toxic derivative of DOX)

daunarubicin

methotrexate

neocarzinostatin

mitomycin C

calicheamicin

vinca alkaloids (vinblastin, vincristine etc)

Enzyme Conjugates for Subsequent Treatment with Pro-Drugs

carboxypeptidase

alkaline phosphatase

### EXAMPLE III

Cell Toxicity of an AMF-Doxorubicin Conjugate

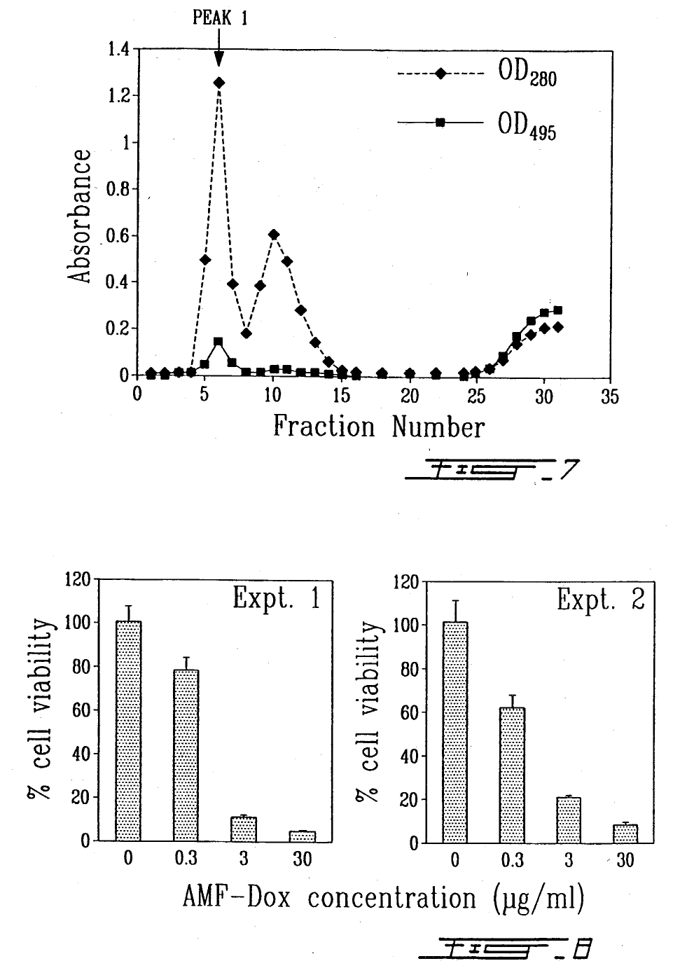

Autocrine motility factor (AMF) is identical to phosphohexose isomerase (PHI) (Watanabe, H. et al. (1996) Cancer Res. 56: 2960-2963) and commercially available PHI (M. W. 60,000) was conjugated to the cellular toxin doxorubicin (M. W. 580) by glutaraldehyde mediated crosslinking. 2 mg of PHI was mixed with 0.4 mg of doxorubicin in the presence of 0.02% glutaraldehyde for 10 minutes at 4° C. Under these conditions minimal crosslinking of PHI to form high molecular weight complexes is observed. The reaction mix was passed over a G25 Sephadex column to separate PHI from the smaller free doxorubicin (FIG. 7). One ml fractions were collected and the major protein peak absorbing at 280 nm (Peak 1) corresponding to PHI also absorbs at 495 nm, the absorbance maximum of doxorubicin, demonstrating that doxorubicin was successfully conjugated to PHI. Free doxorubicin eluted more slowly and corresponds to the peak of 495 nm absorbance after fraction 25.

Fractions 5, 6 and 7 were pooled and concentrated to 400 μl. The concentrated fraction had an OD280 of 2.58 and OD495 of 0.36. Based on the OD280 of a 1 mg/ml solution of PHI and taking into consideration the contribution of doxorubicin absorbance at 280 nm, the approximate concentration of the AMF-Dox fraction was determined to be 3.3 mg/ml.

Rabbit PHI is internalized by murine NIH-3T3 fibroblasts (Benlimame et al., 1998, Molec. Biol. Cell 9: 1773-1786) and the ability of the AMF-doxorubicin conjugate to kill metastatic tumor cells was tested with the B16-F10 metastatic murine melanoma cell line. B16-F10 cells were plated at a density of 4,000 cells/cm2 and after 24 hours in culture AMF-doxorubicin conjugate was added at a concentration of approximately 0.3, 3, or 30 μg/ml (FIG. 8). After 3 (Expt. 1) or 4 days (Expt. 2) the cultures were fixed and cells remaining attached to the substrate counted. The number of cells remaining attached to the substrate is presented relative to control cells to which no AMF-doxorubicin was added. Increasing concentrations of AMF-doxorubicin is associated with progressive toxicity to B16-F10 melanoma cells demonstrating that AMF conjugated to a cellular toxin can kill tumor cells.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

[image]

[image]

[image]

[image]

[image]

[image]

[image]

[image]

[image]

[image]

[image]

[image]

[image]

[image]

[image]

[image]

[image]

[image]

What is claimed is:

1. A therapeutical conjugate to specifically kill motile cells expressing autocrine motility factor receptor, which comprises a first molecule which binds to said autocrine motility factor receptor attached to a second toxic molecule to kill said motile cells.

2. The conjugate of claim 1, wherein said cells are metastatic tumor cells.

3. The conjugate of claim 2, wherein said first molecule is autocrine motility factor (AMF).

4. The conjugate of claim 2, wherein said second molecule is selected from the group consisting of plant toxins, bacterial toxins, fungal toxins, drugs, and enzymes.

5. The conjugate of claim 4, wherein said plant toxin is selected from the group consisting of ricin, abrin, modeccin, viscumin, pokeweed antiviral protein (PAP), saporin, gelonin, momoridin, trichosanthin, barley toxin, and bryodin.

6. The conjugate of claim 4, wherein said bacterial toxin is selected from the group consisting of pseudomonas exotoxin (PE), and diphtheria toxin.

7. The conjugate of claim 4, wherein said fungal toxin is selected from the group consisting of α-sarcin, and restrictocin.

8. The conjugate of claim 4, wherein said drug is selected from the group consisting of doxorubicin, 2-pyrrolinodoxorubicin, daunarubicin, methotrexate, neocarzinostatin, mitomycin C, calicheamicin, and vinca alkaloids.

9. The conjugate of claim 4, wherein said enzyme is selected from the group consisting of carboxypeptidase, and alkaline phosphatase.

10. A method for the manufacture of a medicament to specifically kill cancer cells expressing autocrine motility factor receptor in vitro or in vivo, or both, the method comprising incorporating an effective amount of the conjugate of claim 1 into the medicament.

11. The method of claim 10, wherein said cells in vitro are leukemias purging cells.

12. The method of claim 10, wherein said cells in vivo are metastatic tumor cells.

13. A method to specifically kill cancer cells expressing autocrine motility factor receptor in vitro or in vivo, or both, the method comprising administering an effective amount of the conjugate of claim 4.
